(19) United States
(12) Patent Application Publication
(10) Pub. No.: US 2023/0335224 A1
(43) Pub. Date: Oct. 19, 2023

(54) METHODS AND SYSTEMS FOR STORING GENOMIC DATA IN A FILE STRUCTURE COMPRISING PROTECTION METADATA

(71) Applicant: KONINKLIJKE PHILIPS N.V., EINDHOVEN (NL)

(72) Inventor: Yee Him Cheung, Cambridge, MA (US)

(21) Appl. No.: 18/028,798

(22) PCT Filed: Sep. 29, 2021
(86) PCT No.: PCT/EP2021/076718

§ 371 Date: Mar. 28, 2023

Related U.S. Application Data
(60) Provisional application No. 63/088,061, filed on Oct. 6, 2020.

(51) Int. Cl. G16B 50/50; G16B 50/30; G16B 50/40

(52) U.S. Cl. CPC G16B 50/50; G16B 50/30; G16B 50/40

(57) ABSTRACT

A method (100) comprising: receiving (120) a genomic dataset comprising genomic data of one or more of a plurality of fields or attributes of different data; generating (130) a protection metadata structure for the genomic dataset, comprising one or more of: (i) specifications for selective encryption of one or more data components and regions of genomic data in an annotation table; (ii) specifications for selective signing of one or more data components and regions of genomic data in the annotation table; (iii) user key information; and (iv) access control policy; compressing (140) the genomic data and the protection metadata structure using one or more compression algorithms to generate a compressed genomic dataset and compressed protection metadata structure; and storing (150) the compressed genomic dataset and the compressed protection metadata structure in a container data structure in memory.

### FIELD OF THE DISCLOSURE

The present disclosure is directed generally to methods and systems for storing large quantities of data with associated metadata, and, in particular, to the compression and storage of genomic data.

### BACKGROUND

High-throughput genomic sequencing (HTS) is an important tool for genomics research, and has numerous applications for discovery, diagnosis, and other methodologies. Often, the results of HTS are processed further to obtain higher-level information. The process of aggregating information deduced from single reads and their alignments to the genome into more complex results is generally known as secondary analysis. In most HTS-based biological studies, the output of secondary analysis is usually represented as different types of annotations associated to one or more genomic intervals on the reference sequences.

Indeed, biological studies typically produce genomic annotation data such as mapping statistics, quantitative browser tracks, variants, genome functional annotations, gene expression data and Hi-C contact matrices. These diverse types of downstream genomic data are currently represented in different formats such as VCF, BED, WIG, and many, many more. These formats typically comprise loosely defined semantics, which leads to issues with interoperability, the need for frequent conversions between formats, difficulty in the visualization of multi-modal data, and complicated information exchange, among other issues.

Additionally, the lack of a single format for diverse types of genomic annotation data has stifled work on compression algorithms and has led to the widespread use of general compression algorithms with suboptimum performance. These algorithms do not exploit the fact the annotation data typically comprises of multiple fields (attributes) with different statistical characteristics and instead compress them together. Further, these prior art storage mechanisms lack functional metadata for supporting advanced features such as selective encryption of sensitive information and digital signature of said information.

### SUMMARY OF THE DISCLOSURE

There is a continued need for a unified data format for the efficient representation and compression of diverse genomic annotation data for file storage and data transport. There is a further need for associating and storing metadata with the compressed genomic data to enable selective encryption of sensitive information, as well as digital signature of information.

The present disclosure is directed to inventive methods and systems for storing genomic data within a data structure comprising a file structure, together with functional metadata integrated into the file structure. Various embodiments and implementations herein are directed to a system or method that receives genomic data and stores that genomic data within a data structure comprising a file structure. The genomic data can be any of a wide variety of different genomic data types, including but not limited to genomic variants (VCF), gene expressions, genomic functional annotations (e.g., BED, GTF, GFF, GFF3, GenBank, etc.), quantitative browser tracks (e.g., Wig, BigWig, BedGraph, etc.), and/or chromosome conformation capture (e.g., HiC files, etc.), among many others. A protection metadata structure for the genomic dataset is generated. The protection metadata structure comprises one or more of: (i) specifications for selective encryption of one or more data components and regions of genomic data in an annotation table data; and (ii) specifications for selective signing of one or more data components and regions of genomic data in the annotation table data; (iii) user key information; and (iv) access control policy. The genomic data and protection metadata structure is compressed using a compression algorithm, and the compressed data is then stored in a container data structure in memory.

Generally, a method for storing genomic data within a data structure comprising a file structure is provided. The method includes: receiving a genomic dataset comprising genomic data of one or more of a plurality of fields or attributes of different data; generating a protection metadata structure for the genomic dataset, comprising one or more of: (i) specifications for selective encryption of one or more data components and regions of genomic data in an annotation table; (ii) specifications for selective signing of one or more data components and regions of genomic data in the annotation table; (iii) user key information; and (iv) access control policy; compressing the genomic data and the protection metadata structure using one or more compression algorithms to generate a compressed genomic dataset and compressed protection metadata structure; and storing the compressed genomic dataset and the compressed protection metadata structure in a container data structure in memory.

According to an embodiment, the method further includes the step of encrypting or decrypting, and optionally compressing or decompressing, individual data components and payload blocks of the genomic data to facilitate random access.

According to an embodiment, the method further includes selecting one or more data components or payload blocks of specific regions of the genomic data in an annotation table, comprising an identification of one or more of data component ID, range of row and column index, range of genomic coordinates, and sample ID for the application of encryption and/or digital signature.

According to an embodiment, the method further includes detecting any overlap among the selected data components or regions in the annotation table, and notifying a user of, and/or automatically removing, detected overlap from the selected data components or regions to ensure each data component or payload block is encrypted not more than once.

According to an embodiment, the method further includes ordering, concatenating, and serializing the selected data components and payload blocks in the annotation table for the generation/verification of digital signature.

According to an embodiment, the method further includes extracting all digital signatures generated for the selected data components and/or regions in the annotation table; retrieving a verification key and verifying each of the extracted digital signatures; and presenting the signature information, optionally providing scope of applicability, signer ID and signing date and time, together with the signature information.

According to an embodiment, the method further includes identifying any selected data components and/or regions in an annotation able on which encryption has been applied; authenticating a user that requested data retrieval, and checking whether the user has sufficient access privilege if any part of the selected data components and/or regions is encrypted; and retrieving a decryption key and decrypting each of the encrypted data components and/or regions; optionally performing data integrity verification; and presenting the retrieved data and any associated signature and/or verification results.

According to an embodiment, the method further includes identifying any data components and/or regions being updated that were previously encrypted and/or signed; reapplying encryption on the updated data that were previously encrypted; generating new digital signatures on the updated data to replace the obsolete ones; compressing the updated data components and/or payload blocks as needed; and storing the updated data and/or digital signatures in the annotation table.

According to an embodiment, the method further includes locking of selected data components and payload blocks protected by digital signatures to allow only authenticated users with sufficient access privileges to update the protected data.

According to a second aspect is a system for storing genomic data within a data structure comprising a file structure. The system includes a genomic dataset comprising genomic data of one or more of a plurality of fields or attributes of different data types; a data structure configured to store genomic data; a data compression algorithm; and a processor configured to: (i) generate a protection metadata structure for the genomic dataset, comprising one or more of: (1) specifications for selective encryption of one or more data components and regions of genomic data in an annotation table; (2) specifications for selective signing of one or more data components and regions of genomic data in the annotation table; (3) user key information; and (4) access control policy; (ii) compress, using the data compression algorithm, the genomic data and the protection metadata structure to generate a compressed genomic dataset and compressed protection metadata structure; and (iii) store the compressed genomic dataset and the compressed protection metadata structure in the data structure.

In various implementations, a processor or controller may be associated with one or more storage media (generically referred to herein as “memory,” e.g., volatile and non-volatile computer memory such as RAM, PROM, EPROM, and EEPROM, floppy disks, compact disks, optical disks, magnetic tape, etc.). In some implementations, the storage media may be encoded with one or more programs that, when executed on one or more processors and/or controllers, perform at least some of the functions discussed herein. Various storage media may be fixed within a processor or controller or may be transportable, such that the one or more programs stored thereon can be loaded into a processor or controller so as to implement various aspects as discussed herein. The terms “program” or “computer program” are used herein in a generic sense to refer to any type of computer code (e.g., software or microcode) that can be employed to program one or more processors or controllers.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for packaging genomic data, in accordance with an embodiment.

FIG. 2 is a schematic representation of a genomic data storage system, in accordance with an embodiment.

FIG. 3 is a schematic representation of a data file structure, in accordance with an embodiment.

FIG. 4 is a flowchart of a method for data encryption/decryption, in accordance with an embodiment.

FIG. 5 is a flowchart of a method for data integrity verification, in accordance with an embodiment.

FIG. 6 is a flowchart of a method for data retrieval, in accordance with an embodiment.

FIG. 7 is a flowchart of a method for data updating, in accordance with an embodiment.

### DETAILED DESCRIPTION OF EMBODIMENTS

The present disclosure describes various embodiments of a system and method for storing genomic data and protection metadata within a data structure. Applicant has recognized and appreciated that it would be beneficial to provide a method and system comprising a unified data format for the efficient representation and compression of diverse genomic annotation data. A genomic data storage system receives a genomic dataset comprising one or more of a plurality of fields or attributes of different data types. The system generates a protection metadata structure for the genomic dataset, comprising one or more of: (i) specifications for selective encryption of one or more data components and regions of genomic data in an annotation table; (ii) specifications for selective signing one or more data components and regions of genomic data in the annotation table; (iii) user key information; and (iv) an access control policy. The genomic data and protection metadata are compressed using a compression algorithm, and the compressed data is then stored in a container data structure memory.

Extending a metadata and security framework with stored genomic data provides advanced functionalities for enhancing the management and analysis of the data, which is especially important for large-scale collaborative genomic studies. For example, the methods and systems described or otherwise envisioned herein enables selective encryption and digital signature(s) to be applied only to sensitive information as decided by users, thereby reducing the computational burden and processing overhead for the enforcement of data security and privacy. Another key advantage of integrating functional metadata into the overall file format is that such crucial metadata is organized and readily available as part of the data file, and is not easily lost or misplaced during data transfer and migration. Further, since data security and privacy is designed into the file format rather than being offered through the storage platform or file management software, stronger data protection is achieved. Moreover, with the syntax and processing mechanism of the information and protection metadata clearly defined in the standard, users can expect consistent or similar functionalities and performance from any compliant software.

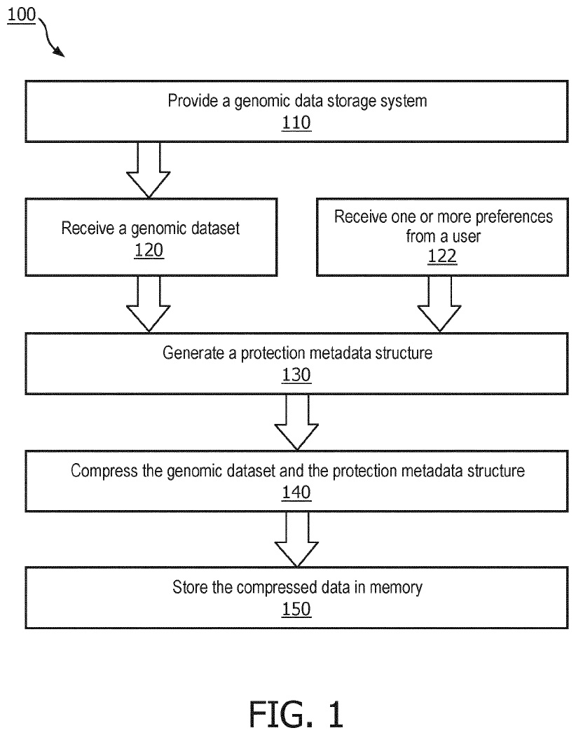

Referring to FIG. 1, in one embodiment, is a flowchart of a method 100 for storing genomic data and associated protection metadata within a data structure comprising a file structure using a genomic data storage system. The methods described in connection with the figures are provided as examples only, and shall be understood not limit the scope of the disclosure. The genomic data storage system can be any of the systems described or otherwise envisioned herein. The genomic data storage system can be a single system or multiple different systems.

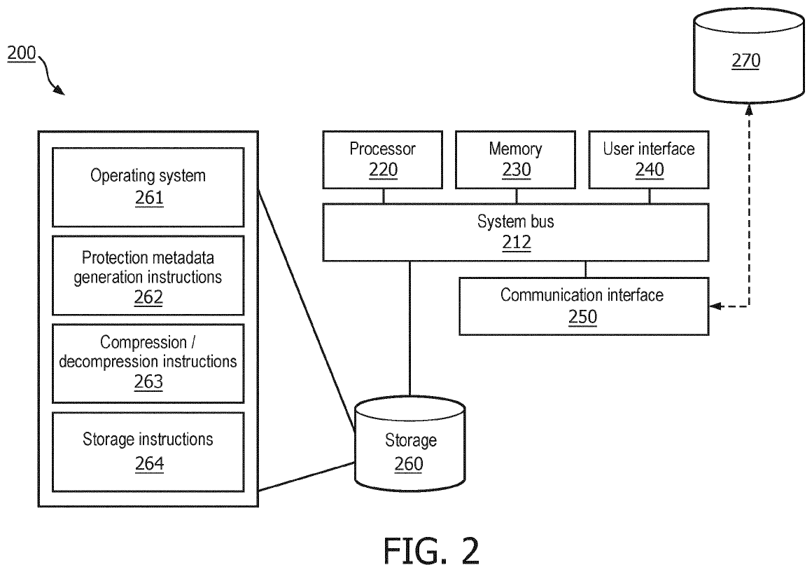

At step 110 of the method, a genomic data storage system is provided. Referring to an embodiment of a genomic data storage system 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the system 200 may be different and more complex than illustrated. Additionally, genomic data storage system 200 can be any of the systems described or otherwise envisioned herein. Other elements and components of genomic data storage system 200 are disclosed and/or envisioned elsewhere herein.

At step 120 of the method, the genomic data storage system receives a genomic dataset comprising genomic data. The genomic data can be any of a wide variety of different genomic data types, including but not limited to genomic variants (VCF), gene expressions, genomic functional annotations (e.g., BED, GTF, GFF, GFF3, GenBank, etc.), quantitative browser tracks (e.g., Wig, BigWig, BedGraph, etc.), and/or chromosome conformation capture (e.g., HiC files, etc.), among many others. The received genomic dataset may comprise genomic data of one type or a plurality of fields or attributes of different data types. The received genomic dataset may be utilized immediately for subsequent steps of the methods described or otherwise envisioned herein, or may be stored for future use by this and other methods. Accordingly, the system may comprise or be in communication with local or remote data storage configured to store the genomic dataset.

At step 130 of the method, the genomic data storage system generates a protection metadata structure for the genomic dataset. The protection metadata structure is configured to enable a wide variety of functionalities, including one or more of support for selective encryption and digital signatures, among other functionalities. The selective encryption (and thus decryption) can be done independently on subsets of the genetic data, thus improving the speed of random access. The selective signing can comprise digital signatures and edit locking for selective portions of data components and/or genomic data.

According to an embodiment, the protection metadata structure for the genomic dataset comprises one or more of: (i) specifications for selective encryption of one or more data components and regions of genomic data in an annotation table; (ii) specifications for selective signing of one or more data components and regions of genomic data in the annotation table; (iii) user key information; and (iv) an access control policy.

The generated protection metadata structure may be utilized immediately for subsequent steps of the methods described or otherwise envisioned herein, or may be stored for future use by this and other methods. Accordingly, the system may comprise or be in communication with local or remote data storage configured to store the genomic dataset and annotation table. Notably, some or all of the protection metadata structure may be encrypted as described or otherwise envisioned herein.

At optional step 122 of the method, the system receives a user input such as through a user interface of the genomic data storage system. The input can be, for example, one or more user preferences, such as an encryption selection and/or a digital signature. For example, the user can designate genomic data and/or annotation table data for encryption. The user can also provide digital signature information for some or all of the genomic data and/or annotation table data.

At step 140 of the method, the genomic data storage system compresses the genomic data and the protection metadata structure using a compression algorithm to generate a compressed genomic dataset. The compression algorithm can be any algorithm, method, or process for data transformation and compression, including but not limited to the compression algorithms and methods described or otherwise envisioned herein. The compression algorithm can be a single compression algorithm or multiple compression algorithms.

At step 150 of the method, the compressed genomic dataset, together with the protection metadata structure is stored in a container data structure, such as an annotation table, in memory. The memory may be any memory capable of receiving and storing the compressed data. The memory may be associated with the genomic data storage system, or may be in direct or indirect wired and/or wireless communication with the genomic data storage system. The memory may be a local or a remote memory. The memory may be a cloud-based memory. Many other storage mechanisms and devices are possible.

Therefore, according to an embodiment, the system comprises protection metadata that is extended to support the selective encryption and signing of annotation table data. Thus, the system includes a URI structure for referencing specific data fields and block/chunk payloads for data protection. The system can also comprise centralized storage of encryption and signature parameters and data that improves the efficiency of data security enforcement.

According to one embodiment of a genomic data storage system, the system processes a received genomic dataset, extracts a plurality of attributes from the genomic dataset, and then breaks each attribute down into a plurality of chunks of a predetermined size. The chunks are indexed in a master index of the data structure, with lookup data for each of the plurality of chunks. Each chunk is individually compressed with a compression algorithm, and is then stored within the allocated location of a chunk structure data of the data structure. Thus, the data structure is configured such that each of the plurality of chunks can be decompressed individually. Further, the data structure is configured such that the genomic data type, the attributes, chunk size, and the compression algorithm can each be modified without changing the file structure of the data structure.

According to an embodiment, the system enables selective encryption and decryption. The selective encryption/decryption can be performed on independently on each block/chunk payload, thus improving the speed of random access. The system can utilize an encrypted flag in the block header to indicate if a block payload is encrypted or not. The system can ensure each block payload is encrypted not more than once by validating the URIs in EncryptionParameters elements. To access data of an annotation table with encryption parameters defined, all encrypted regions need to be identified by resolving the URIs in the EncryptionParameters elements. Any encrypted data being accessed should be decrypted for presentation.

According to an embodiment, the system enables selective digital signing. The selective digital signing can include rules for the concatenation of specific data fields and block/chunks payloads for the generation of digital signatures. A region can be protected by multiple digital signatures by different users. A boolean editLock attribute can be added to SignatureParameters to indicate if the signed data should be locked from editing. If editLock is turned on, editing of the signed data is only allowed for authenticated users. After making changes, the old signatures should be discarded or re-generated. To update data of an annotation table with signature parameters defined, all signed regions protected by edit locking need to be identified by resolving the URIs in the EncryptionParameters elements.

According to an embodiment, each of the metadata components consisting of the whole XML, document can be encrypted and signed with the inclusion of table ID, table name, table version, last update user ID and last update time to increase the uniqueness of the signature value to prevent it from being reused.

Selective Encryption/Decryption

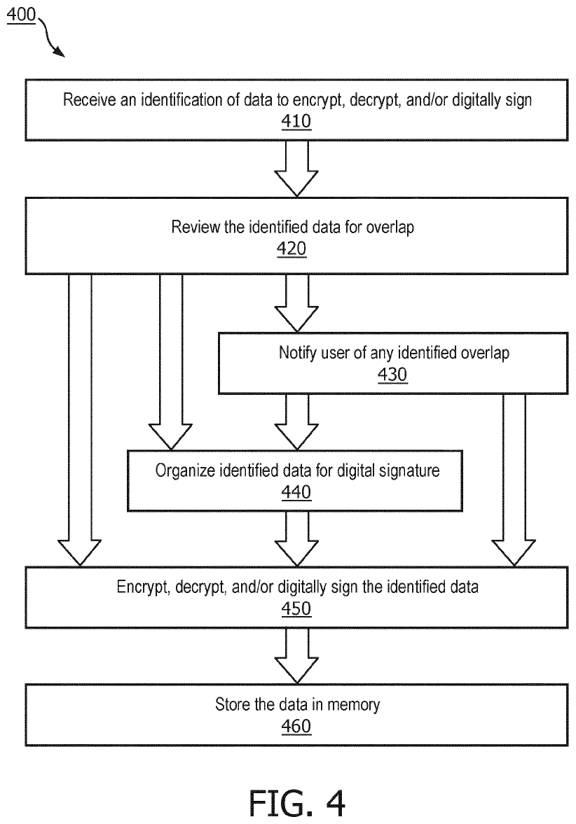

Referring to FIG. 4, in one embodiment, is a method 400 for selective encryption and/or decryption, and/or digital signature, of genomic data or other data within the genomic dataset. According to an embodiment, the annotation table is structured to enable the selective encryption and/or decryption of genetic data, components, or annotation access unit payloads within the annotation table.

At step 410 of the method, the genomic data storage system receives an identification of data to encrypt or decrypt, and/or to digitally sign. The data identified for encryption, decryption, and/or digital signature can be any of the data in the protection metadata structure or the genomic dataset. The identified data can comprise individual data components and/or payload blocks of the genomic data, which can significantly facilitate access to that data. The identification can be received from a user of the genomic data storage system, and can be received via a user interface of the system. Accordingly, the system facilitates selection of data for protection of data security and/or privacy via the user interface.

According to an embodiment, one or more data components or payload blocks of specific regions of the genomic data in an annotation table identified for encryption, decryption, and/or digital signature is identified by specifying one or more of a combination of one or more of data component ID, range of row and column index, range of genomic coordinates, and sample ID. Many other methods for the identification of the data are possible.

At step 420 of the method, the genomic data storage system analyzes the identification of data to determine whether there is any overlap among the selected data components or regions in an annotation table. If there is any overlap detected, the system notifies the user at step 430 and/or automatically removes the detected overlap(s) from the selected data components or regions to ensure each data component or payload block is encrypted not more than once. If there are no overlaps detected, the method may progress to the next step. According to an embodiment, the user is notified via a user interface of the system.

At step 440 of the method, the selected data components and payload blocks in the annotation table is ordered, concatenated, and/or serialized for the generation/verification of digital signature. The selected data can be ordered, concatenated, and/or serialized using any method suitable to prepare the data for digital signature.

At step 450 of the method, the genomic data storage system encrypts, decrypts, or digitally signs the data in the protection metadata structure or the genomic dataset identified for encryption, decryption, and/or digital signature. The identified data comprises individual data components and/or payload blocks of the genomic data. According to an embodiment, the system optionally compresses or decompresses the data while encrypting, decrypting, and/or digitally signing. The encrypted, decrypted, and/or digitally signed data can then be stored in memory at step 460 of the method.

Data Integrity Verification

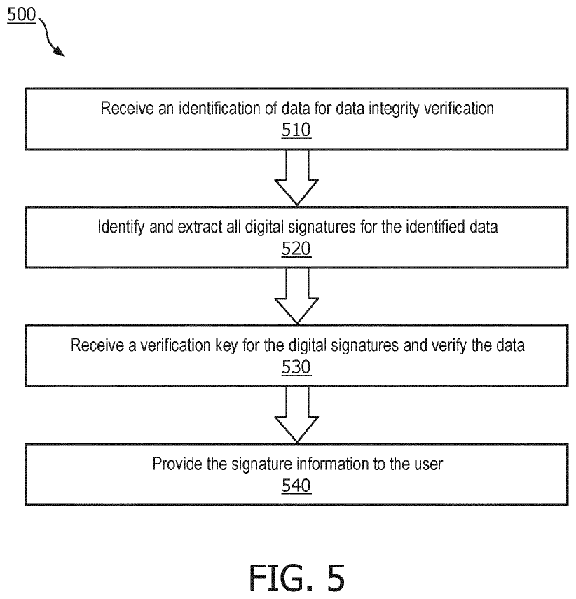

Referring to FIG. 5, in one embodiment, is a method 500 for data integrity verification. According to an embodiment, the annotation table is structured to enable the selective verification of the integrity of genetic data, components, or annotation access unit payloads within the annotation table.

At step 510 of the method, the genomic data storage system receives an identification of data for integrity verification. The data identified for integrity verification can be any of the data in the protection metadata structure or the genomic dataset. The identified data can comprise individual data components and/or payload blocks of the genomic data. The identification can be received from a user of the genomic data storage system, and can be received via a user interface of the system. Accordingly, the system facilitates selection of data for integrity verification via the user interface.

At step 520 of the method, the genomic data storage system identifies and extracts all digital signatures generated for the selected data components and/or regions in the annotation table.

At step 530 of the method, the system retrieves a verification key, which may be obtained from one of a plurality of sources, and verifies each of the identified and extracted digital signatures. Verification using a verification key can be performed using any of a wide variety of methods.

At step 540 of the method, the system provides the signature information to a user, such as through a user interface of the genomic data storage system. The signature information may be any information associated with the digital signature and data, including but not limited to the scope of applicability, signer ID and signing date and time, and other information.

Data Retrieval

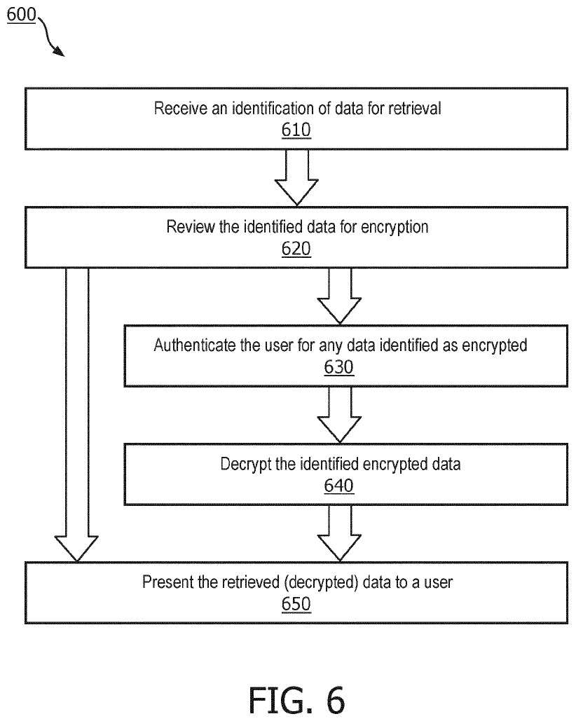

Referring to FIG. 6, in one embodiment, is a method 600 for data retrieval. According to an embodiment, the annotation table is structured to enable the selective retrieve of data, which can be any of the genetic data, components, or annotation access unit payloads within the annotation table.

At step 610 of the method, the genomic data storage system receives an identification of data for retrieval. The data identified for retrieval can be any of the data in the protection metadata structure or the genomic dataset. The identified data can comprise individual data components and/or payload blocks of the genomic data. The identification can be received from a user of the genomic data storage system, and can be received via a user interface of the system. Accordingly, the system facilitates selection of data for retrieval via the user interface.

At step 620 of the method, the genomic data storage system reviews the selected data components and/or regions in the annotation able to identify any such data that has been encrypted.

At step 630 of the method, if any of the selected data is encrypted, the genomic data storage system authenticates the user that requested the data retrieval in order to determine whether the user has sufficient access privilege to access the encrypted data.

At step 640 of the method, the genomic data storage system retrieves a decryption key and decrypts each of the encrypted data components and/or regions. The system may optionally perform data integrity verification during or after encryption, as described or otherwise envisioned herein.

At step 650 of the method, the genomic data storage system provides the retrieved data to a user, such as through a user interface of the genomic data storage system. The retrieved data may be accompanied by any associated signature and/or verification results, among other possible data or information.

Data Update

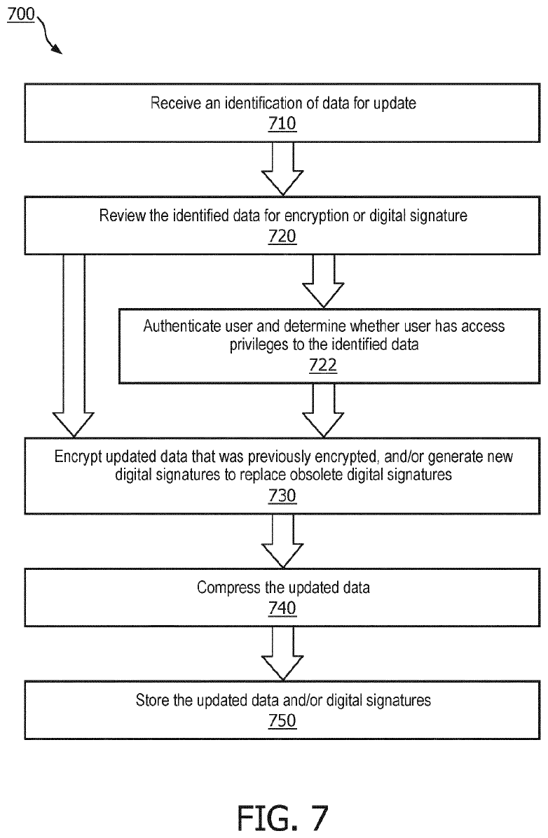

Referring to FIG. 7, in one embodiment, is a method 700 for updating data in the stored genomic data file. According to an embodiment, the annotation table is structured to enable the selective updating of data, which can be any of the genetic data, components, or annotation access unit payloads within the annotation table.

At step 710 of the method, the genomic data storage system receives an identification of data for update. The data identified for update can be any of the data in the protection metadata structure or the genomic dataset. The identified data can comprise individual data components and/or payload blocks of the genomic data. The identification can be received from a user of the genomic data storage system, and can be received via a user interface of the system. Accordingly, the system facilitates selection of data for updating via the user interface.

At step 720 of the method, the genomic data storage system reviews the selected data components and/or regions in the annotation able to identify any such data that has been encrypted or digitally signed.

At optional step 722 of the method, for any data that is identified as being locked from editing, the genomic data storage system authenticates the user and determines whether they have sufficient access privileges to that data.

At step 730 of the method, the genomic data storage system reapplies encryption on the updated data that were previously encrypted, and/or generates new digital signatures on the updated data to replace the obsolete digital signatures. Accordingly, the user or system can optionally choose to lock the selected data components and payload blocks protected by digital signatures to allow only authenticated users with sufficient access privileges to update the protected data.

At step 740 of the method, the genomic data storage system compresses the updated data components and/or payload blocks. At step 750 of the method, the system stores the updated data and/or digital signatures in the annotation table.

Genomic Data Storage Structure and Data Format

The genomic data storage structure in which the received genomic data and associated annotation table is packaged may take any of a wide variety of formats. Although a specific format is described with reference to an embodiment, below, it is understood that this is just one example of a data structure that may be utilized by the genomic data storage system described or otherwise envisioned herein. Similarly, the format of the data within the genomic data storage structure may take any of a wide variety of formats. Although a specific format is described with reference to an embodiment, below, it is understood that this is just one example of a data format that may be utilized by the genomic data storage system described or otherwise envisioned herein.

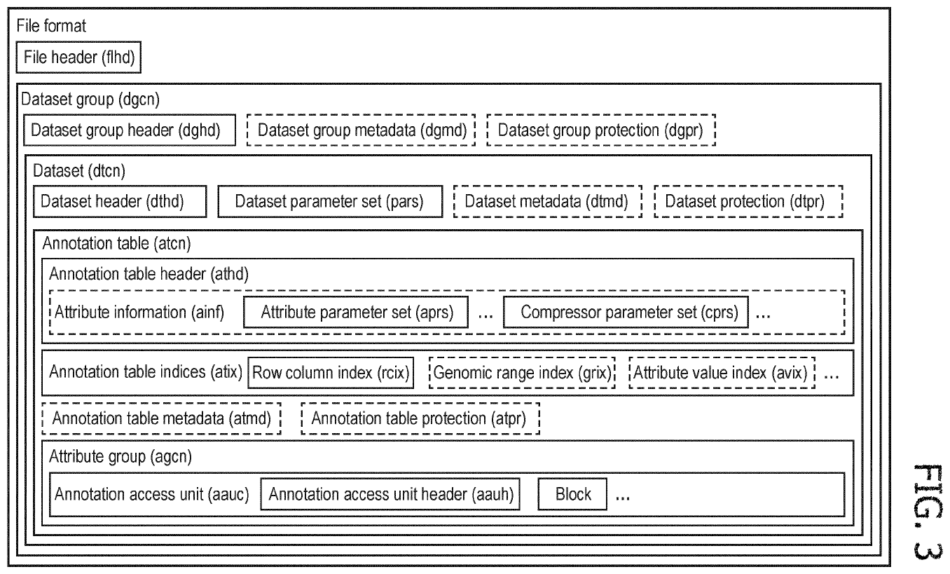

Referring to FIG. 3 is an embodiment of a top-level container hierarchy for a genomic dataset and associated annotation table. In this format, the top-level container boxes of File, Dataset Group, and Dataset are utilized. The Dataset comprises an Annotation Table (atcn) with the data. In FIG. 3, all container boxes, including Dataset Group (dgcn), Dataset (dtcn), Annotation Table (atcn), Attribute Group (agcn), and Annotation Access Unit (aauc), can exist in multiple instances. For example, the “ . . . ” symbol behind a box indicates there can be multiple instances of that specific box structure.

According to an embodiment, the information and protection metadata can be stored respectively in the Annotation Table Metadata and Annotation Table Protection data structures, which are enclosed in gen_info boxes in KLV (Key, Length, Value) format with syntax as follows, although other syntax is possible:

| struct gen_info { | |
|---|---|
| c(4) | Key; |
| u(64) | Length; |
| u(8) | Value[ ]; |

}

According to an embodiment, the Key field specifies the type of the data structure in a four-character code, which is “atmd” for Annotation Table Metadata and “atpr” for Annotation Table Protection. The Length field specifies the number of bytes composing the entire gen_info structure, including all three fields Key, Length and Value. The syntaxes of the Value fields of Annotation Table Metadata and Annotation Table Protection are defined respectively in TABLE 1 and TABLE 2.

TABLE 1
Syntax of Annotation Table Metadata

| Syntax | Key | Type | Remarks |
|---|---|---|---|
| annotation_table_metadata { | atmd | | |
| dataset_group_ID | | u(8) | Dataset group identifier |
| dataset_ID | | u(16) | Dataset identifier |
| AT_ID | | u(8) | Annotation table |
| | | | identifier |
| ATMD_general_exist | | u(1) | Flag for the existence of |
| | | | general information |
| if (ATMD_general_exist) { | | | |
| ATMD_general_size | | u(16) | Size in number of bytes |
| | | | of general information |
| ATMD_general( ) | | u(v) | General information of |
| | | | the annotation table |
| } | | | |
| ATMD_analytics_exist | | u(1) | Flag for the existence of |
| | | | analytics specifications |
| if (ATMD_analytics_exist) { | | | |
| ATMD_analytics_size | | u(16) | Size in number of bytes |
| | | | of analytics |
| | | | specifications |
| ATMD_analytics( ) | | u(v) | Analytics specifications |
| } | | | |
| ATMD_linkages_exist | | u(1) | Flag for the existence of |
| | | | linkage information |
| if (ATMD_linkages_exist) { | | | |
| ATMD_linkages_size | | u(16) | Size in number of bytes |
| | | | of linkage information |
| ATMD_linkages( ) | | u(v) | Linkages to other data |
| | | | objects |
| } | | | |
| ATMD_history_exist | | u(1) | Flag for the existence of |
| | | | access history |
| if (ATMD_history_exist) { | | | |
| ATMD_history_size | | u(16) | Size in number of bytes |
| | | | of access history |
| ATMD_history( ) | | u(v) | Access history of the |
| | | | annotation table |
| } | | | |
| reserved | | u(4) | Trailing zeros for byte |
| | | | alignment |
| } | | | |

TABLE 2
Syntax of Annotation Table Protection

| Syntax | Key | Type | Remarks |
|---|---|---|---|
| annotation_table_protection { | atpr | | |
| dataset_group_ID | | u(8) | Dataset group identifier |
| dataset_ID | | u(16) | Dataset identifier |
| ann_table_ID | | u(8) | Annotation table |
| | | | identifier |
| AT_protection_value( ) | | | Protection metadata |
| } | | | |

Annotation Table Protection Metadata

According to an embodiment, the Annotation Table Protection gen_info box with key “atpr” holds the parameters for data protection, which includes encryption and digital signature, and the rules for access control that apply to the information metadata and block payloads within an annotation table. It is in the form of an XML document, with a root element “AnnotationTableProtection”. The document is compressed by the LZMA algorithm and the compressed bytes are stored in the AT_protection_value( ) element of the gen_info box. The output of the decoding process is an XML document with the root node AnnotationTableProtection, which consists of four main components:

(1) Any number of “KeyTransportAES” elements, each defines a key identified by the keyName element and its key transport parameters. More details on the key transport parameters and mechanisms can be found in subclause 7.2.4 of ISO/IEC 23092-3.

(2) Any number of “EncryptionParameters” elements, each has a mandatory encryptedLocations attribute that specifies a URI to reference a data target, and the associated cipher algorithm and key. In particular, the following rules apply: (i) the IV element shall be present; (ii) the TAG element shall not be present; and (iii) the configurationID attribute shall be present. If an access unit belongs to the collection resolved by the URI, the AccessUnitEncryptionParameters element of the access unit protection shall contain one or more wrappedKey elements each referring to the configurationID. The key associated to the EncryptionParameters shall allow to unwrap the corresponding wrappedKey.

(3) Any number of “SignatureParameters” elements of SignatureType, each holds the signature value and its associated parameters, including the signature method and one or multiple reference elements, each with a URI attribute for specifying a URI to reference a data target. Detached, Enveloped and Enveloping signatures are supported. If decryption is required, signature verification shall be performed before decryption.

(4) A “privacy rules” element that contains a valid access control policy specified according to the OASIS, eXtensible Access Control Markup Language (XACML) Version 3.0 specification. The privacy rules specify who can execute a given action and under which conditions. More details can be found in subclause 7.3 of ISO/IEC 23092-3

The protection metadata of a data container has a limited scope of applicability. In general, its parameters are used for encrypting or signing the information metatdata at the same level, or the protection metadata of the container(s) at the next lower level, and its policy rules are used for controlling access to any resources within the container. In the case of Annotation Table Protection, it also governs the protection of the block payloads in the enclosed annotation access units.

According to an embodiment, there are at least three types of data protection targets, including but not limited to the following: (1) specific elements in an XML document for metadata and protection gen_info boxes; (2) data fields in metadata and protection gen_info boxes; and (3) block payloads in annotation access units containing data from selected regions of an annotation table, among other targets.

Regarding the first type of target that involves specific XML elements, the syntax and processing rules for data encryption and digital signature recommended by the W3C Working Group are directly applied. For encryption, the choice of providing certain plaintext values as encrypted contents can be offered by including elements of type EncryptedData. In this scenario, the mechanism to transmit the knowledge of the keys shall be established through another channel. For digital signing, a signature element, as defined in the xmldsig schema, can be included for each data object to be signed.

Regarding the second and third types of target that involves data fields in gen_info boxes and block payloads in annotation access units, the XML, elements KeyTransportAES, EncryptionParameters and SignatureParameters in protection metadata are used with basically the same syntax as described in ISO/IEC 23092-3. In particular, details on the key retrieval and encryption parameters can be found in subclauses 7.2.4 and 7.2.5 of ISO/IEC 23092-3. There are, however, a few aspects of the data encryption and signature framework that need to be extended or modified for annotation table data and will be discussed in the subsequent sections: (1) the URI structure for identifying the specific data fields and block payloads to be protected; (2) the encryption and decryption processes of the block payloads in annotation access units; (3) the rules for the concatenation of specific data fields and block payloads for the generation of digital signatures.

According to an embodiment, to protect the confidentiality and integrity of the Annotation Table Protection metadata, which might contain sensitive security information, its encryption and signing can be enabled by specifying its URI and relevant parameters in the protection metadata of the enclosing dataset. With proper access control settings, only authenticated and authorized users can read, update or sign on the protection metadata. If signing is enabled, only the latest signature is kept. To further prevent the protection metadata and its corresponding signature from being replaced by an obsolete previous version, optional LastUpdateUser element of type string and LastUpdateTime element of type dateTime can be included in the XML document for encryption and signing, with the corresponding update record, including the last update user and time, entered into the secure access history in Annotation Table Metadata. Similarly, optional TableID, TableName and TableVersion elements of type string can be included to ensure that the protection metadata can only be used for the table of specific ID, name and version. In this case, the protection metadata has to be updated with proper encryption and signing whenever the table ID or version is changed.

URI (Universal Resource Identifier) Structure

A URI structure is defined for referencing specific gen_info box components or annotation access unit payloads within an annotation table, in order to enable their selective encryptions or signings. The following are some general rules on the URI syntax: (i) text within curly brackets, including the curly brackets themselves, shall be replaced by some alphanumeric sequence compliant with the description of each entry; (ii) in a semantics table, parameters marked by an asterisk (*) are mandatory, otherwise, they are optional; (iii) an optional field can be left blank if it is not used for selecting the target, i.e. the target covers all values of the field; (iv) a URI can be contracted by removing any redundant trailing fields and slashes.

The keys and parameters for encrypting and signing the bytes of the element AT_protection_value( ) in Annotation Table Protection can be specified within the protection metadata of the upper-level Dataset container. TABLE 3 comprises a URI structure for this purpose.

TABLE 3
ann_table/{ann_table_id}/protection

| Parameter | Type | Semantics |
|---|---|---|
| ann_table_id* | unsigned | Identifier of the annotation table of |
| | integer | which the protection metadata is to be |
| | 0-255 | encrypted or signed. Its value shall be |
| | | one of the ann_table_IDs listed in the |
| | | Dataset Header. |

In Annotation Table Protection, the following URI structure can be used for referencing specific data fields of the metadata gen_info box within the same annotation table, as shown in TABLE 4.

TABLE 4
metadata/{md_fields}

| Parameter | Type | Semantics |
|---|---|---|
| md_fields | st(v) | The specific fields in Annotation Table Metadata to be |
| | | encrypted or signed. It can be a single or combination |
| | | of the following values concatenated by a pound |
| | | symbol “#”: |
| | | “general” that refers to the field ATMD_general( ) |
| | | “analytics” that refers to the field ATMD_analytics( ) |
| | | “history” that refers to the field ATMD_history( ) |
| | | “linkages” that refers to the field ATMD_linkages( ) |
| | | If the field is blank or specified as “all”, the URI |
| | | refers to all the elements. |

Note that for the encryption of metadata fields, each field is encrypted independently using the associated encryption parameters, with the ciphertext replacing the original bytes. For digital signature, the bytes of the selected fields are concatenated in the same order as defined in the syntax of Annotation Table Metadata and signing is performed on the concatenated bytes. The resultant signature is then stored as an XML signature element in the protection box.

In Annotation Table Protection, the URI structure in TABLE 5 can be used for referencing specific regions of an annotation table on which data protection is to be applied. The URI can correspond to any block payloads in annotation access units that overlap with the target region, which can be specified through a combination of genomic coordinates, row/column indices, sample IDs or attribute values.

TABLE 5
AT_region/{AG_classes}/{range_type_1} = {range_1}/{range_type_2} =
{range_2}/desc_ids = {desc_IDs}/attr_ids = {attr_IDs}

| Parameter | Type | Semantics |
|---|---|---|
| AG_classes* | st(v) | One or multiple attribute group classes that contain the attribute |
| | | data to be protected. Each attribute group class corresponds to the |
| | | field attribute_group_class in the header of one of the attribute |
| | | groups in the annotation table. The string could be a single class |
| | | value, a hyphenated range of class values or a concatenation of |
| | | single or range of class values separated by the pound symbol |
| | | “#”. For example, “1-3#5” covers the attribute group classes 1, 2, |
| | | 3 and 5. If the field is left blank or specified as “all”, all attribute |
| | | groups are covered. |
| range_type_1, | st(v) | The type of attribute range values being used for specifying the |
| range_type_2 | | target region. The possible values include: |
| | | “range_genome” for genomic coordinates |
| | | “range_row_idx” for 1-based indices associated with the rows |
| | | of the main attribute group |
| | | “range_col_idx” for 1-based indices associated with the |
| | | columns of the main attribute group |
| | | “range_idx” for 1-based indices if the main attribute group is |
| | | one dimensional |
| | | “range_desc:{AG_class}:{desc_ID}” for a range based on the |
| | | value of a descriptor specified by its containing attribute group |
| | | class (AG_class) and descriptor ID (desc_ID). Note that |
| | | AG_class values 1 and 2 refer respectively to the auxiliary |
| | | attribute groups associated with the rows and columns of the |
| | | main attribute group. |
| | | “range_attr:{AG_class}:{attr_ID}” for a range based on the |
| | | value of an attribute specified by its containing attribute group |
| | | class (AG_class) and attribute ID (attr_ID). Note that |
| | | AG_class values 1 and 2 refer respectively to the auxiliary |
| | | attribute groups associated with the rows and columns of the |
| | | main attribute group. |
| | | The second range is only needed when the target region is two |
| | | dimensional. In that case the ranges must be respectively for the |
| | | rows and columns, and if the row/column range is not specified, |
| | | the target region covers all the rows/columns. |
| range_1, | st(v) | Different string formats should apply depending on the range |
| range_2 | | type: |
| | | For “range_genome” type, its format should be one or |
| | | multiple instances of “{seq_name}:{pos_from}-{pos_to}” |
| | | concatenated by the pound symbol “#”, where seq_name is the |
| | | sequence/chromosome ID, and (pos_from, pos_to) are the |
| | | start and end positions of the target region on the |
| | | sequence/chromosome. If the target is a single nucleotide |
| | | position, the part “-{pos_to}” can be omitted. If the target |
| | | includes the whole sequence/chromosome, the part |
| | | “:{pos_from}-{pos_to}” can be omitted. |
| | | For “range_row_idx”, “range_col_idx” and “range_idx” types, |
| | | its format should be one or multiple instances of |
| | | “{index_from}-{index_to}” concatenated by the pound |
| | | symbol “#”, where (index_from, index_to) are the 1-based |
| | | start and end indices of the target region. If the target consists |
| | | of only a single row/column, the part “-{index_to}” can be |
| | | omitted. |
| | | For “range_desc” and “range_attr” types, its format should be |
| | | one or multiple instances of “{value_from}-{value_to}” |
| | | concatenated by the pound symbol “#”', where the target |
| | | region is bounded by the first element matching value_from |
| | | and the next nearest element matching value to in the |
| | | specified descriptor/attribute. Except for the first instance, the |
| | | to and from values of any subsequent instances are matched |
| | | against the descriptor/attribute elements after the previous |
| | | identified interval. Note that if the descriptor/attribute is of the |
| | | string type, the values should be enclosed by double quotes. |
| desc_IDs | st(v) | The IDs of the descriptors whose block payloads are to be |
| | | protected. It should be a concatenated string of a single or a |
| | | hyphenated range of descriptor IDs separated by the pound |
| | | symbol “#”. If left blank or specified as “all”, the URI covers all |
| | | descriptors belonging to the attribute group classes specified in |
| | | AG_classes. If specified as “none”, all descriptors are excluded. |
| attr_IDs | st(v) | The IDs of the attributes whose block payloads are to be |
| | | protected. It should be a concatenated string of a single or a |
| | | hyphenated range of attribute IDs separated by the pound symbol |
| | | “#”. If left blank or specified as “all”, the URI covers all attributes |
| | | belonging to the attribute group classes specified in AG_classes. |
| | | If specified as “none”, all attributes are excluded. |

Assuming a two-dimensional annotation table, such as a variant call file, with genomic coordinates for the rows and sample IDs for the columns, the following are two examples of the URI structure and the targets they represent:
(1) AT_region/0/range_genome=chr1#chr2:1-100000/range_attr:2:1=“Sample 1”-“Sample 10” refers to the block payloads of all descriptors and attributes that: (i) belong to the main attribute group of class 0; (ii) contain data in the genomic regions of chromosome 1 or the first 100,000 nucleotides of chromosome 2, and (iii) correspond to the columns between “Sample 1” and “Sample 10”, as defined in the column-associated attribute (AG_class=2) of ID 1, in the annotation table.
(2) AT_region/all/range_row_idx=10000-20000/range_col_idx:1-10/attr_ids=1-5 refers to the block payloads of all descriptors and the attributes of IDs from 1 to 5 that contain data in the rectangular region bounded by rows 10,000-20,000 and columns 1-10 in the data of the main attribute group.

According to an embodiment, for the encryption of block payloads in annotation access units, each block payload referenced by the URI can be encrypted independently using the associated encryption parameters, with the cyphertext replacing the original bytes. For digital signature, signing is performed on the concatenated bytes of the referenced block payloads and the resultant signature is then stored as a XML, signature element in the protection box.

Selective Encryption and Decryption

According to an embodiment, the following is an encryption/decryption process when the value of the encryptedLocations element of the XML EncryptionParameters element matches with the URI structure beginning with “AT_region” for referencing a target region in an annotation table:
1. Look up the tiles that overlap with the target region using precomputed indexing data in Annotation Table Indices.
2. Locate the corresponding block payloads in the annotation access units.
3. Retrieve from the EncryptionParameters element: (i) the key; and (ii) the configurationID.
4. Retrieve from the AccessUnitEncryptionParameters element present in the associated Annotation Access Unit (AAU) protection box: (i) the cipher (possible values are listed in Table 14 of ISO/IEC 23092-3); (ii) the wrappedKey instances matching the configurationID (retrieved in the previous step); (iii) auinIV, if the AAU contains an AAU information box; (iv) auinTAG, if the AAU contains an AAU information box and the cipher uses GCM mode; (v) aublockIV; and (vi) aublockTAG if the cipher uses GCM mode.
5. Encrypt/Decrypt each block payload identified in step 2 individually using the wrappedKey instance (obtained in the previous step) associated with the attribute/descriptor ID (for tile-contiguity mode) or tile index(es) (for attribute-contiguity mode) that uniquely identifies the block payload in the AAU.
6. If the encrypted/decrypted data is to be stored, it can simply replace the original bytes of the block payload, since the lengths of the ciphertext and plaintext are the same for both the CTR and GCM encryption modes supported by the framework. The encrypted flag in the block header should also be updated accordingly (0 for plaintext, 1 for ciphertext).

Measures can be taken to ensure that each block payload cannot be encrypted more than once. When a new set of encryption parameters is set up, the URI referencing the target region should be checked against the URIs in any existing EncryptionParameters elements. If there are any overlapping target regions, then check whether or not the same key was used for the encryption of each overlapping block payload. If it is true, the new set of encryption parameters is valid and encryption can be applied on the non-overlapping block payloads in the target region. If different keys were used, the URI for the new target region should be modified, e.g. by breaking up into multiple URIs, so as to avoid any overlaps with the existing encrypted regions. This can ensure that an encrypted region is always associated with only one encryption key in the protection metadata.

For accessing data of an annotation table with encryption parameters defined, all encrypted regions need to be identified by resolving the URIs in the EncryptionParameters elements. If a block payload is found to be located in one of the encrypted regions, or its encrypted flag in the block header is set to 1, then decryption should be applied on the block payload using the key associated with the URI for the encrypted region.

The encryption/decryption process for specific data fields in gen_info boxes is similar. The main difference is that another URI structure “metadata/{md_fields}” should be used to reference one or multiple data fields to be individually encrypted and replaced by the generated ciphertext. As in the case of block payloads, each data field cannot be encrypted more than once. Measures should be taken to ensure a data field is referenced by the URI of at most one EncryptionParameters element.

Selective Digital Signature

The generation and verification of digital signatures for data in an annotation table comprises a set of rules for the concatenation of bytes of the selected data fields or block payloads referenced by the newly introduced URI structures for Annotation Table. Hash and signature algorithms are applied on the concatenated bytes to generate a digital signature to be stored in the corresponding SignatureParameters element in protection metadata.

For signing a set of metadata fields selected by a URI of the format “metadata/{md_fields}”, the bytes are concatenated in the same order as the fields are defined in the syntax of Annotation Table Metadata.

For signing the block payloads in annotation access units selected by a URI of the format “AT_region/{AG_classes}/{range_type_1}={range_1}/{range_type_2}={range_2}/desc_ids={desc_IDs}/attr_ids={attr_IDs}”, the following two rules for the concatenation of bytes should apply:
(1) In attribute-contiguity mode, where each annotation access unit contains payloads of all tiles (blocks of rows and columns in an annotation table) associated with a descriptor/attribute: (i) within an access unit, the block_payload( ) bytes of the selected tiles are concatenated in increasing order of their tile index. For two-dimensional data, if column_major_tile_order equals 1, the ordering is first by column and then by row indices; otherwise, the ordering is first by row and then by column indices; (ii) within an attribute group, the payload bytes of the selected access units from the previous step are then concatenated in increasing order of first their descriptor ID and then their attribute ID; and (iii) the payload bytes of the selected attribute groups from the previous step are then concatenated in increasing order their attribute group class.
(2) In tile-contiguity mode, where each annotation access unit contains payloads of all descriptors/attributes associated with a tile of an annotation table: (i) within an access unit, the block_payload( ) bytes of the selected descriptors/attributes are concatenated in increasing order of first their descriptor ID and then their attribute ID; (ii) within an attribute group, the payload bytes of the selected access units from the previous step are then concatenated in increasing order of their tile index. For two-dimensional data, if column_major_tile_order equals 1, the ordering is first by column and then by row indices; otherwise, the ordering is first by row and then by column indices; and (iii) the payload bytes of the selected attribute groups from the previous step are then concatenated in increasing order of their attribute group class.

Unlike the case of encryption, which can only be applied at most once on a region, multiple digital signatures by different users can be applied on the same region. Therefore, it is permissible to have URIs in multiple SignatureParameters elements that reference regions overlapping with each other.

To further protect the signed data from unauthorized changes, a boolean editLock attribute can be added to SignatureParameters to indicate if the signed data should be locked from editing. The enforcement of edit locking requires the signature parameters to be securely stored, so that any changes to them can only be made by authorized users.

If editLock is turned on, editing of the signed data is only allowed for authenticated users with higher levels of access rights than all the current signees, or authenticated users having access to all the keys for the current signatures. After making changes to the signed and locked data, and passing the authentication and authorization processes, the signature parameters should be updated by either discarding any associated SignatureParameters elements or regenerating their signatures if the keys are available. An authorized user can also create new SignatureParameters elements to ensure data integrity in selected regions with or without edit locks. If editLock is turned off, the signed data and its associated signature parameters can be changed by any users.

For modifying data of an annotation table with signature parameters defined, all signed regions protected by edit locking need to be identified by resolving the URIs in the SignatureParameters elements. If data update is requested in one of the signed and locked regions, it will only be approved on passing the user authentication and authorization processes, with old signatures discarded/re-generated and new signatures generated as desired by the user.

The advantages of this encryption and digital signature framework are manifold. First, it allows data to be protected only in selected regions of an annotation table that contain sensitive data, thus reducing the overall processing time for data security enforcement. Second, it supports fast random access to the encrypted data. Since encryption is performed on each block payload independently, only the selected block payloads need to be decrypted and decompressed, thus improving the speed of response for random access. Third, with all encryption and signature parameters and data centrally stored in Annotation Table Protection, it improves the efficiency of decryption, integrity verification, and data protection for data access and editing.

Referring to FIG. 2, in one embodiment, is a schematic representation of a system 200 for storing genomic data. System 200 may be any of the systems described or otherwise envisioned herein, and may comprise any of the components described or otherwise envisioned herein.

According to an embodiment, system 200 comprises one or more of a processor 220, memory 230, user interface 240, communications interface 250, and storage 260, interconnected via one or more system buses 212. In some embodiments, the hardware may include a genomic data database 270. It will be understood that FIG. 2 constitutes, in some respects, an abstraction and that the actual organization of the components of the system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RANI is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (MC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RANI), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms and/or instructions to carry out one or more functions or steps of the methods described or otherwise envisioned herein. For example, processor 220 may comprise one or more of annotation table generation instructions 262, compression/decompression instructions 263, and/or storage instructions 264.

According to an embodiment, annotation table generation instructions 262 direct the system to generate or modify an annotation table within the file structure for the genomic dataset. The annotation table is configured to enable a wide variety of functionalities, including one or more of support for selective encryption and digital signatures.

According to an embodiment, compression/decompression instructions 263 direct the system to compress the genomic data along with the associated annotation table. The compression algorithm can be any algorithm, method, or process for data compression. The compression instructions may also comprise decompression instructions for decompression stored data.

According to an embodiment, storage instructions 264 direct the system to store the compressed genomic data and associated annotation table. The system may comprise or be in communication with local or remote data storage configured to store the genomic dataset and annotation table.

The processing of a genomic dataset, the generation of an annotation table, and compression/decompression of the genomic data and annotation table comprises millions or billions of calculations, something the human mind is not equipped to perform, even with pen and pencil. Indeed, the genomic dataset alone comprises millions of pieces of information. For example, next-generation DNA sequencing data comprises reads that number in the 100s of millions or even billions.

Further, the methods described herein significantly improve the speed and functionality of a genomic storage system. For example, by implementing the methods described herein, the genomic storage system comprises an annotation table with protection metadata configured for: (i) selective encryption of annotation table data and/or genomic data; and (ii) selective signing of annotation table data and/or genomic data. Prior art systems cannot provide this functionality, and therefore are inferior systems. Accordingly, the methods described herein significantly improve the speed and functionality of a genomic storage system.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

1. A method for storing genomic data within a data structure comprising a file structure, the method comprising:
receiving a genomic dataset comprising genomic data of one or more of a plurality of fields or attributes of different data;
generating a protection metadata structure for the genomic dataset, comprising one or more of: (i) specifications for selective encryption of one or more data components and regions of genomic data in an annotation table; (ii) specifications for selective signing of one or more data components and regions of genomic data in the annotation table; and (iii) user key information;
compressing the genomic data and the protection metadata structure using one or more compression algorithms to generate a compressed genomic dataset and compressed protection metadata structure; and
storing the compressed genomic dataset and the compressed protection metadata structure in a container data structure in memory.

2. The method of claim 1, further comprising the step of encrypting or decrypting, and optionally compressing or decompressing, individual data components and payload blocks of the genomic data to facilitate random access.

3. The method of claim 1, further comprising the step of selecting one or more data components or payload blocks of specific regions of the genomic data in an annotation table, comprising an identification of one or more of data component ID, range of row and column index, range of genomic coordinates, and sample ID for the application of encryption and/or digital signature.

4. The method of claim 3, further comprising the step of detecting any overlap among the selected data components or regions in the annotation table, and notifying a user of, and/or automatically removing, detected overlap from the selected data components or regions to ensure each data component or payload block is encrypted not more than once.

5. The method of claim 3, further comprising the steps of ordering, concatenating, and serializing the selected data components and payload blocks in the annotation table for the generation/verification of digital signature.

6. The method of claim 3, further comprising the steps for data integrity verification:
extracting all digital signatures generated for the selected data components and/or regions in the annotation table;
retrieving a verification key and verifying each of the extracted digital signatures; and
presenting the signature information, optionally providing scope of applicability, signer ID and signing date and time, together with the signature information.

7. The method of claim 1, further comprising the steps for data retrieval:
identifying any selected data components and/or regions in an annotation able on which encryption has been applied;
authenticating a user that requested data retrieval, and checking whether the user has sufficient access privilege if any part of the selected data components and/or regions is encrypted; and
retrieving, if authenticating determines that the user has sufficient access privilege, a decryption key and decrypting each of the encrypted data components and/or regions;
optionally performing data integrity verification; and
presenting the retrieved data and any associated signature and/or verification results.

8. The method of claim 1, further comprising the steps for data update:
identifying any data components and/or regions being updated that were previously encrypted and/or signed;
reapplying encryption on the updated data that were previously encrypted;
generating new digital signatures on the updated data to replace the obsolete ones;
compressing the updated data components and/or payload blocks as needed; and
storing the updated data and/or digital signatures in the annotation table.

9. The method of claim 8, further comprising locking of selected data components and payload blocks protected by digital signatures to allow only authenticated users with sufficient access privileges to update the protected data.

10. A system for storing genomic data within a data structure comprising a file structure, the system comprising:
a genomic dataset comprising genomic data of one or more of a plurality of fields or attributes of different data types;
a data structure configured to store genomic data;
a data compression algorithm; and
a processor configured to: (i) generate a protection metadata structure for the genomic dataset, comprising one or more of: (1) specifications for selective encryption of one or more data components and regions of genomic data in an annotation table; (2) specifications for selective signing of one or more data components and regions of genomic data in the annotation table; and (3) user key information; (ii) compress, using the data compression algorithm, the genomic data and the protection metadata structure to generate a compressed genomic dataset and compressed protection metadata structure; and (iii) store the compressed genomic dataset and the compressed protection metadata structure in the data structure.

11. The system of claim 10, wherein the processor is further configured to further encrypt or decrypt, and optionally compress or decompress, individual data components and payload blocks of the genomic data to facilitate random access.

12. The system of claim 10, wherein the processor is further configured to receive a selection of one or more data components or payload blocks of specific regions of the genomic data in an annotation table, comprising an identification of one or more of data component ID, range of row and column index, range of genomic coordinates, and sample ID for the application of encryption and/or digital signature.

13. The system of claim 10, wherein the processor is further configured to extract all digital signatures generated for the selected data components and/or regions in the annotation table; retrieve a verification key and verifying each of the extracted digital signatures; and present the signature information, optionally providing scope of applicability, signer ID and signing date and time, together with the signature information.

14. The system of claim 10, wherein the processor is further configured to identify any selected data components and/or regions in an annotation able on which encryption has been applied; authenticate a user that requested data retrieval, and checking whether the user has sufficient access privilege if any part of the selected data components and/or regions is encrypted; and retrieve, if authenticating determines that the user has sufficient access privilege, a decryption key and decrypting each of the encrypted data components and/or regions; optionally perform data integrity verification; and present the retrieved data and any associated signature and/or verification results.

15. The system of claim 10, wherein the processor is further configured to identify any data components and/or regions being updated that were previously encrypted and/or signed; reapply encryption on the updated data that were previously encrypted; generate new digital signatures on the updated data to replace the obsolete ones; compress the updated data components and/or payload blocks as needed; and store the updated data and/or digital signatures in the annotation table.
